(19) United States
(12) Patent Application Publication
(10) Pub. No.: US 2023/0233553 A1
(43) Pub. Date: Jul. 27, 2023

(54) A NEW COMBINATION THERAPY FOR THE TREATMENT OF FGFR3- RELATED SKELETAL DISEASE

(71) Applicants: INSERM (INSTITUT NATIONAL DE LA SANTÉ ET DE LA RECHERCHE MÉDICALE), Paris (FR); FONDATION IMAGINE, Paris (FR); Université Paris Cité, Paris (FR); Assistance Publique-Hôpitaux de Paris (APHP), Paris (FR); University of Connecticut, Storrs, CT (US)

(72) Inventors: Laurence LEGEAI-MALLET, Paris (FR); Laurinda JAFFE, Farmington, CT (US); Leia SHUHAIBAR, Farmington, CT (US)

(21) Appl. No.: 18/003,783

(22) PCT Filed: Jun. 28, 2021
(86) PCT No.: PCT/EP2021/067744

§ 371 Date: Dec. 29, 2022

(30) Foreign Application Priority Data
Jun. 29, 2020 (EP) 20305724.5

(51) Int. Cl. A61K 31/496; A61K 38/22; A61P 19/08

(52) U.S. Cl. CPC A61K 31/496; A61K 38/2242; A61P 19/08

(57) ABSTRACT

Activating mutations in fibroblast growth factor receptor 3 (FGFR3) and inactivating mutations in the natriuretic peptide receptor 2 (NPR2) guanylyl cyclase both result in decreased production of cyclic GMP (cGMP) and severe short stature, causing achondroplasia and acromesomelic dysplasia type Maroteaux, respectively. In attempt to find a new therapeutic approach for FGFR3-related skeletal disease, the inventors showed that a combination of a NPR2 agonist (e.g. BMN-111) and a phosphatase inhibitor (e.g. LB-100) significantly increases the length of the Fgfr3^{Y367C/+} femurs compared to Fgfr3^{+/+} femurs and improves the whole growth plate cartilage. The present invention thus relates to the use of a NPR2 agonist (e.g. BMN-111) and a phosphatase inhibitor (e.g. LB-100) for the treatment of FGFR3-related skeletal disease (e.g. achondroplasia).

### FIELD OF THE INVENTION

The present invention relates to the treatment or prevention of skeletal disorders developed by patients that display abnormal increased activation of the fibroblast growth factor receptor 3 (FGFR3). The present invention also relates a new combination therapy of FGFR3-related skeletal disease comprising a NPR2 agonist (e.g. BMN-111) and a phosphatase inhibitor (e.g. LB-100).

### BACKGROUND OF THE INVENTION

Achondroplasia (ACH), the most common form of dwarfism, is due to a gain of function mutation in the fibroblast growth factor receptor type 3 (FGFR3) gene (Rousseau et al., 1994; Shiang et al., 1994). FGFR3 is expressed in growth plate cartilage and bone, which explains the bone anomalies observed in patients with ACH. The characteristic features of these patients are short arms and legs, macrocephaly, hypoplasia of the midface, lordosis, spinal stenosis, and low bone mineral density (Ornitz and Legeai-Mallet, 2017). Research over the last decade and the generation of Fgfr3-specific mouse models have highlighted the role of FGFR3 during bone growth. In the absence of Fgfr3, the most prominent phenotype of the mice is overgrowth, thus indicating that FGFR3 is a negative regulator of bone growth (Colvin et al., 1996; Deng et al., 1996). Conversely, mice expressing a Fgfr3-activating mutation develop dwarfism, and have reduced linear growth and impaired endochondral ossification with reduced chondrocyte proliferation and reduced hypertrophic differentiation (Naski et al., 1998; Chen et al., 1999; Li et al., 1999; Pannier et al., 2010). A complex intracellular network of signals including FGFR3 mediates this skeletal phenotype. Activating mutations in FGFR3 lead to upregulated FGFR3 protein expression (Legeai-Mallet et al., 2004) and to increased activity of several downstream intracellular signaling pathways including MAPK, PI3K/AKT, PLCy and STATs (Ornitz and Itoh, 2015).

During development, the rate of longitudinal bone growth is determined by chondrocyte proliferation and differentiation and is regulated by several secreted growth factors and endocrine factors, including parathyroid hormone-like peptide, Indian Hedgehog, bone morphometric proteins, transforming growth factor (3, insulin like growth factor, and C-type-natriuretic peptide (CNP) (Long and Ornitz, 2013). CNP and its receptor, the guanylyl cyclase natriuretic peptide receptor 2 (NPR2) also known as guanylyl cyclase B, are expressed in chondrocytes as well in osteoblasts and are recognized as important regulators of longitudinal bone growth and bone homeostasis. NPR2 possesses guanylyl cyclase activity that leads to synthesis of cyclic guanosine-3′,5′-monophosphate (cGMP), and dysregulation of this pathway is responsible for skeletal disorders. In clinical studies, inactivating mutations of NPR2 were found to cause extreme short stature, namely acromesomelic dysplasia type Maroteaux (Maroteaux et al., 1971; Tamura et al., 2004; Bartels et al., 2004; Khan et al., 2012; Geister et al., 2013; Nakao et al., 2015). Conversely, heterozygous NPR2 gain of function mutations cause tall stature (Miura et al., 2014), and overexpression of CNP due to a balanced translocation is responsible for overgrowth and bone anomalies (Bocciardi et al., 2007; Moncla et al., 2007). Animal models with Npr2 loss of function mutations or with disruption of the CNP gene (Nppc) show severe dwarfism, while overstimulation of CNP and NPR2 causes overgrowth disorders. All of these data support a key role of the CNP/NPR2 signaling pathway for normal growth (Chusho et al., 2001; Tsuji and Kunieda, 2005).

Recent studies have indicated that among its diverse signaling effects, activation of FGFR3 results in reduced phosphorylation and activity of NPR2 in the growth plate, thus lowering cGMP and opposing bone growth (Robinson et al., 2017; Shuhaibar et al., 2017). Mechanistically, the CNP-induced increase in bone growth is due at least in part to cGMP counteracting the FGF-induced decrease in chondrocyte cell division by inhibiting the ERK pathway that is stimulated by FGF (Krejci et al., 2005; Ozasa et al., 2005). Synthesis of cGMP by NPR2 requires extracellular binding of C-type natriuretic peptide (CNP) (Potter, 1998), and CNP or a hydrolysis-resistant CNP analog, known as BMN-111 or vosoritide, increases bone growth in mouse models of achondroplasia, demonstrating a significant recovery of bone growth mediated by NPR2/cGMP signaling (Yasoda et al., 2004; Lorget et al., 2012). These preclinical studies were confirmed in human, and vosoritide (a CNP analog) is currently in clinical development, with phase two results showing additional height gain in achondroplasia patients treated with vosoritide (Savarirayan et al., 2019). NPR2 activity also requires phosphorylation of the NPR2 protein on multiple sites (Potter, 2011). FGF-induced dephosphorylation of NPR2 reduces its guanylyl cyclase activity, by way of a PPP family phosphatase, suggesting that a phosphatase inhibitor could enhance bone growth if applied together with CNP (Robinson et al., 2017; Shuhaibar et al., 2017).

Here the inventors investigated the possible use of the PPP family phosphatase inhibitor LB-100 (D'Arcy et al., 2019), for which phase one clinical trials have indicated safety and efficacy (Chung et al., 2017). In studies of animal cancers, LB-100 has been shown to enhance the responses to immunotherapy, CAR-T cell therapy, and tyrosine kinase inhibitors (Ho et al., 2018; Lai et al., 2018; Cui et al., 2020). The inventors find that LB-100 counteracts the FGF-induced dephosphorylation and inactivation of NPR2, complementing the CNP stimulation and promoting bone growth in a mouse model of achondroplasia.

### SUMMARY OF THE INVENTION

The present invention relates to methods and pharmaceutical compositions for the treatment of FGFR3-related skeletal disease. In particular, the present invention is defined by the claims.

### DETAILED DESCRIPTION OF THE INVENTION

Activating mutations in fibroblast growth factor receptor 3 (FGFR3) and inactivating mutations in the natriuretic peptide receptor 2 (NPR2) guanylyl cyclase both result in decreased production of cyclic GMP (cGMP) and severe short stature, causing achondroplasia and acromesomelic dysplasia type Maroteaux, respectively. Previously the inventors showed that an NPR2 agonist BMN-111 increases bone growth in a mouse model of achondroplasia, and found that in growth plate chondrocytes, FGFR3 signaling decreases NPR2 activity by dephosphorylating the NPR2 protein. Here the inventors tested whether a phosphatase inhibitor could enhance bone growth and counteract the effect of overactive FGFR3 as seen in achondroplasia. Using an ex vivo imaging system with mice expressing a FRET sensor to measure changes in cGMP production in chondrocytes of living tibias from newborn mice, the inventors showed that the PPP family phosphatase inhibitor LB-100 counteracts the FGF-induced dephosphorylation and inactivation of NPR2. In ex vivo experiments with a mouse model of achondroplasia (Fgfr3^{Y67C/+}), the inventors found that LB-100 in combination with BMN-111 increases the rate of bone growth by —25% compared with BMN-111 alone. The results suggest that a phosphatase inhibitor could be used together with an NPR2 agonist to enhance cGMP production as a therapy for achondroplasia.

The present invention relates to a method of treatment of FGFR3-related skeletal diseases in a patient need thereof comprising in administering a therapeutically effective amount of a combination of a phosphatase inhibitor and a NPR2 agonist.

The inventors show that a combination of a NPR2 agonist (e.g. BMN-111) and a phosphatase inhibitor (e.g. LB-100) significantly increases the length of the Fgfr3^{Y367C/+} femurs compared to Fgfr3^{+/+} femurs and improves the whole growth plate cartilage.

The present invention shows the use of a combination (BMN-111+LB100) to increase, promote, stimulate, raise, elevate, improve, enhance the bone growth, whole growth plate cartilage.

The present invention relates to a method of improving the bone growth in a patient need thereof comprising in administering a therapeutically effective amount of a combination of a phosphatase inhibitor and a NPR2 agonist.

The present invention relates to a method of increasing the whole growth plate cartilage in a patient need thereof comprising in administering a therapeutically effective amount of a combination of a phosphatase inhibitor and a NPR2 agonist.

The present invention relates to a method of increasing the bone growth in a patient need thereof comprising in administering a therapeutically effective amount of a combination of a phosphatase inhibitor and a NPR2 agonist.

The present invention relates to a method of improving the whole growth plate cartilage in a patient need thereof comprising in administering a therapeutically effective amount of a combination of a phosphatase inhibitor and a NPR2 agonist.

As used herein, the term “subject” denotes a mammal, such as a rodent, a feline, a canine, and a primate. Particularly, the subject according to the invention is a human. Particularly, the subject according to the invention is an adult. Particularly, the subject according to the invention is a child. Particularly, the subject according to the invention is a teenager. Particularly, the subject according to the invention is a new born. Particularly, the subject according to the invention is a subject having less than 15 years old, less than 10 years old. Particularly, the subject according to the invention is a subject is having 1 to 15 years old. Particularly, the subject according to the invention is having 2 to 10 years old. Particularly, the subject according to the invention is having 1, 2, 3, 4, 5, 6, 7, 8, 9, 10, 11, 12, 13, 14, 15 years old.

As used herein, the terms “FGFR3”, “FGFR3 tyrosine kinase receptor” and “FGFR3 receptor” are used interchangeably throughout the specification and refer to all of the naturally-occurring isoforms of FGFR3.

As used herein, the term “FGFR3-related skeletal disease” is intended to mean a skeletal disease that is caused by an abnormal increased activation of FGFR3, in particular by expression of a constitutively active mutant of the FGFR3 receptor, in particular a constitutively active mutant of the FGFR3 receptor as described above.

As used herein, the expressions “constitutively active FGFR3 receptor variant”, “constitutively active mutant of the FGFR3” or “mutant FGFR3 displaying a constitutive activity” are used interchangeably and refer to a mutant of said receptor exhibiting a biological activity (i.e. triggering downstream signaling), and/or exhibiting a biological activity which is higher than the biological activity of the corresponding wild-type receptor in the presence of FGF ligand. A constitutively active FGFR3 variant according to the invention is in particular chosen from the group consisting of (residues are numbered according to their position in the precursor of fibroblast growth factor receptor 3 isoform 1—806 amino acids long—): a mutant wherein the serine residue at position 84 is substituted with lysine (named herein below S84L); a mutant wherein the arginine residue at position 248 is substituted with cysteine (named herein below R200C); a mutant wherein the arginine residue at position 248 is substituted with cysteine (named herein below R248C); a mutant wherein the serine residue at position 249 is substituted with cysteine (named herein below S249C); a mutant wherein the proline residue at position 250 is substituted with arginine (named herein below P250R); a mutant wherein the asparagine residue at position 262 is substituted with histidine (named herein below N262H); a mutant wherein the glycine residue at position 268 is substituted with cysteine (named herein below G268C); a mutant wherein the tyrosine residue at position 278 is substituted with cysteine (named herein below Y278C); a mutant wherein the serine residue at position 279 is substituted with cysteine (named herein below S279C); a mutant wherein the glycine residue at position 370 is substituted with cysteine (named herein below G370C); a mutant wherein the serine residue at position 371 is substituted with cysteine (named herein below S371C); a mutant wherein the tyrosine residue at position 373 is substituted with cysteine (named herein below Y373C); a mutant wherein the glycine residue at position 380 is substituted with arginine (named herein below G380R); a mutant wherein the valine residue at position 381 is substituted with glutamate (named herein below V381E); a mutant wherein the alanine residue at position 391 is substituted with glutamate (named herein below A391E); a mutant wherein the asparagine residue at position 540 is substituted with Lysine (named herein below N540K); a mutant wherein the termination codon is eliminated due to base substitutions, in particular the mutant wherein the termination codon is mutated in an arginine, cysteine, glycine, serine or tryptophane codon (named herein below X807R, X807C, X807G, X807S and X807W, respectively); a mutant wherein the lysine residue at position 650 is substituted with another residue, in particular with methionine, glutamate, asparagine or glutamine (named herein below K650M, K650E, K650N and K650Q). Typically, a constitutively active FGFR3 variant according to the invention is K650M, K650E or Y373C mutant.

In some embodiments, the FGFR3-related skeletal diseases are FGFR3-related chondrodysplasias and FGFR3-related craniosynostosis.

In some embodiments, the FGFR3-related skeletal osteochondrodysplasias correspond to an inherited or to a sporadic disease.

As used herein, the term “FGFR3-related skeletal dysplasias” includes but is not limited to thanatophoric dysplasia type I, thanatophoric dysplasia type II, hypochondroplasia, achondroplasia and SADDAN, severe achondroplasia with developmental delay and acanthosis nigricans.

In particular, the FGFR3-related skeletal disease is achondroplasia (ACH).

In some embodiments, the FGFR3-related skeletal osteochondrodysplasia is caused by expression in the subject of a constitutively active FGFR3 receptor variant such as defined above.

In some embodiments, the FGFR3-related chondrodysplasia is an achondroplasia caused by expression of the G380R constitutively active mutant of the FGFR3 receptor.

In some embodiments, the FGFR3-related chondrodysplasia is a hypochondroplasia caused by expression of the N540K, K650N, K650Q, S84L, R200C, N262H, G268C, Y278C, S279C, V381E, constitutively active mutant of the FGFR3 receptor.

In some embodiments, the FGFR3-related chondrodysplasia is a thanatophoric dysplasia type I caused by expression of a constitutively active mutant of the FGFR3 receptor chosen from the group consisting of R248C, S248C, G370C, S371C; Y373C, X807R, X807C, X807G, X807S, X807W and K650M FGFR3 receptors.

In some embodiments, the FGFR3-related chondrodysplasia is a thanatophoric dysplasia type II caused by expression of the K650E constitutively active mutant of the FGFR3 receptor.

In some embodiments, the FGFR3-related chondrodysplasia is a severe achondroplasia with developmental delay and acanthosis nigricans caused by expression of the K650M constitutively active mutant of the FGFR3 receptor.

In some embodiments, the FGFR3-related craniosynostosis corresponds to an inherited or to a sporadic disease. In some embodiments, the FGFR3-related craniosynostosis is Muenke syndrome caused by expression of the P250R constitutively active mutant of the FGFR3 receptor or Crouzon syndrome with acanthosis nigricans caused by expression of the A391E constitutively active mutant of the FGFR3 receptor.

As used herein, the term “bone growth” relates to the increase in the diameter of bones by the addition of bony tissue at the surface of bones.

As used herein, the term “whole growth plate cartilage” relates to the areas of new bone growth in children and teens. They are made up of cartilage, a rubbery, flexible material (the nose, for instance, is made of cartilage). Most growth plates are near the ends of long bones. Cartilaginous endplate are thin layers of cartilage found adjacent to the intervertebral disc, bridging the disc with the vertebral bone. Long bones are bones that are longer than they are wide.

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “preventing” intends characterizing a prophylactic method or process that is aimed at delaying or preventing the onset of a disorder or condition to which such term applies.

As used herein, the term “C-type natriuretic peptide receptor” has its general meaning in the art and refers to a receptor for C-type natriuretic peptide. Three types of natriuretic peptide receptors have been identified on which natriuretic peptides act. They are all cell surface receptors and designated:
guanylyl cyclase-A (GC-A) also known as natriuretic peptide receptor-A (NPRA/ANPA) or NPR1
guanylyl cyclase-B (GC-B) also known as natriuretic peptide receptor-B (NPRB/ANPB) or NPR2
natriuretic peptide clearance receptor (NPRC/ANPC) or NPR3

As used herein, the term “natriuretic peptide receptor 2”, “NPR-B” or “NPR2” are used interchangeably throughout the specification and has a single membrane-spanning segment with an extracellular domain that binds the ligand. The intracellular domain maintains two consensus catalytic domains for guanylyl cyclase activity. Binding of a natriuretic peptide induces a conformational change in the receptor that causes receptor dimerization and activation. The binding of C-type natriuretic peptide (CNP) to its receptor causes the conversion of GTP to cGMP and raises intracellular cGMP.

As used herein, the term “gene” has its general meaning in the art and refers a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

As used herein the term “agonist” refers to an agent (i.e. a molecule) for which a natural or synthetic compound has a biological effect to increase the activity of for example NPR2.

As used herein the term “NPR2 agonist” refers to an agonist of NPR2 which is a molecule that has a biological effect to increase the activity of NPR2 receptor. Preferably, the NPR2 agonist according to the invention acts through direct interaction with the NPR2 receptor.

In some embodiments, the treatment consists of administering to the subject a NPR2 agonist.

In some embodiment, the NPR2 agonist is BMN-111.

As used herein, the term “BMN-111” also known as “Vosoritide” has the following formula C₁₇₆H₂₉₀N₅₆O₅₁S₃ and the following CAS Number: 1480724-61-5.

In some embodiment, the NPR2 agonist is ASB-20123.

As used herein, the term “ASB-20123” refers to the full-length 22-amino acids of human CNP-22 fused to the 17-amino acids on the C-terminus region of human ghrelin, and the single amino acid is substituted in its ghrelin region.

In some embodiment, the NPR2 agonist is “CNP-53”.

As used herein, the term “CNP-53” refers to a C-type natriuretic peptide with 53 amino acids.

As used herein, the term “phosphatase” has its general meaning in the art and refers to an enzyme that catalyzes the hydrolysis of a phosphomonoester, removing a phosphate moiety from the substrate. Water is split in the reaction, with the —OH group attaching to the phosphate ion, and the H+ protonating the hydroxyl group of the other product. The net result of the reaction is the destruction of a phosphomonoester and the creation of both a phosphate ion and a molecule with a free hydroxyl group. Phosphatase enzymes recognize and catalyze a wider array of substrates and reactions.

As used herein, the term “protein phosphatase” (PP) has its general meaning in the art and refers to a phosphatase enzyme that removes a phosphate group from the phosphorylated amino acid residue of its substrate protein. Protein phosphorylation is one of the most common forms of reversible protein posttranslational modification (PTM), with up to 30% of all proteins being phosphorylated at any given time. Protein phosphatases (PPs) are the primary effectors of dephosphorylation and can be grouped into three main classes based on sequence, structure and catalytic function:
The largest class of PPs is the phosphoprotein phosphatase (PPP) family comprising PP1 (or PPP1), PP2A (PPP2), PP2B (PPP3), PP4, PP5, PP6 and PP7, and the protein phosphatase Mg2+- or Mn2+-dependent (PPM) family, composed primarily of PP2C.
The protein Tyr phosphatase (PTP) super-family forms the second group.
The aspartate-based protein phosphatases forms the third

As used herein the term “inhibitor” refers to an agent (i.e. a molecule) which inhibits or blocks the activity of FGFR3. For instance, an antagonist of FGFR3 refers to a molecule which inhibits or blocks the activity of the FGFR3 receptor. Preferably, the FGFR3 antagonists according to the invention act through direct interaction with the FGFR3 receptor.

In some embodiments, the treatment consists of administering to the subject a phosphatase inhibitor.

In some embodiment, the phosphatase inhibitor is LB-100.

As used herein, the term “LB-100” refers to a general water soluble protein phosphatase 2A (PP2A) inhibitor has the following formula C₁₃H₂₀N₂O₄ and the following CAS Number: 1632032-53-1.

The inhibitor according to the invention is capable of inhibiting or eliminating the functional activation of the FGFR3 receptor in vivo and/or in vitro. The inhibitor may inhibit the functional activation of the FGFR3 receptor by at least about 10%, preferably more by 20 at least about 30%, preferably by at least about 50%, preferably by at least about 70, 75 or 80%, still preferably by 85, 90, 95, or 100%.

As used herein the terms “administering” or “administration” refer to the act of injecting or otherwise physically delivering a substance as it exists outside the body (e.g. a phosphatase inhibitor and/or a NPR2 agonist) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

As used herein, the term “combination” is intended to refer to all forms of administration that provide a first drug together with a further (second, third . . . ) drug. The drugs may be administered simultaneously, separately or sequentially and in any order. According to the invention, the drug is administered to the subject using any suitable method that enables the drug to reach the chondrocytes of the bone growth plate. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the growing bone.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The present invention also relates to a therapeutically effective amount of a combination of a phosphatase inhibitor and a NPR2 agonist for use in the treatment of FGFR3-related skeletal disease (e.g. achondroplasia)

The present invention also relates to a therapeutically effective amount of a combination of LB-100 and BMN-111 for use in the treatment of FGFR3-related skeletal disease (e.g. achondroplasia).

In a particular embodiment, the invention relates to a i) phosphatase inhibitor and ii) NPR2 agonist for simultaneous, separate or sequential use in the treatment of FGFR3-related skeletal diseases (e.g. achondroplasia).

In a particular embodiment, the invention relates to i) LB-100 and ii) BMN-111 for simultaneous, separate or sequential use in the treatment of FGFR3-related skeletal diseases (e.g. achondroplasia).

The phosphatase inhibitor and/or the NPR2 agonist as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. LB-100 and BMN-111 act synergistically to stimulate growth in fetal femurs from Fgfr3^{Y367C/+} mice.(A) Diagram showing sites of action of LB-100 and BMN-111. (B) Representative photographs of fetal femurs from E16.5 day old Fgfr3+/+ (wildtype) and Fgfr3^{Y367+} mice, before (D0) and after a 6 day (D6) culture with the indicated treatments. The upper dashed line indicates the groups compared in C-F. (C, D) Measurements of growth in bone length (C) and area (D), showing that in Fgfr3^{Y67C/+} bones, BMN-111+LB-100 increases growth more than BMN-111 alone. (E, F) Measurements of growth in bone length (E) and area (F) for Fgfr3+/+ bones, showing that for Fgfr3+/+ bones, BMN-111+LB-100 does not increase growth more than BMN-111 alone. For all experiments, the concentration of BMN-111 was 0.1 μM, and the concentration of LB-100 was 10 μM. Symbols in graphs C-E indicate individual bones (n=4−10). Bars represent mean ±SEM and data were analyzed by unpaired two-tailed t-tests. Asterisks indicate significant differences (p<0.05) between indicated groups; n.s. indicates no significant difference (p>0.05).

FIG. 2. Dual action of LB-100 and BMN-111 improves chondrocyte differentiation in growth plates of ex-vivo cultured Fgfr3^{Y367C/+} femurs. Mean area of individual hypertrophic chondrocytes in proximal growth plates of femurs treated (n=4−6 bones measured for each condition, with 54-137 cells measured for each bone). Data were analyzed by one-way ANOVA followed by the Holm-Sidak multiple comparison test (*p<0.05, **p<0.01, ****p<0.0001).

FIG. 3. Graphic representation of naso-anal, femur, tibia, ulna and humerus length and % of bone growth of Fgfr3^{Y367C/+} mice treated with subcutaneous injection of LB100 (1 mg.kg−1 body weight)+BMN111 (800 ug.kg−1 body weight)

FIG. 4. Graphic representation of skull and foramen magnum length and foramen magnum area and % of growth of Fgfr3^{Y367C/+} mice treated with subcutaneous injection of LB100 (1 mg.kg−1 body weight)+BMN111 (800 ug.kg−1 body weight)

### EXAMPLE 1: EX-VIVO

### Material & Methods

### Mice

Two mouse lines were used for this study: cGi500 (Thunemann et al., 2013) and Fgfr3^{Y367C/+} (Pannier et al., 2009; Lorget et al., 2012). The cGi500 mice were provided by Robert Feil. The use of the cGi500 mice for monitoring cGMP levels in the growth plate has been verified by ELISA measurements (Shuhaibar et al., 2017).

### Reagents

Reagents were obtained from the sources listed in Supplementary Table I. BMN-111 was synthesized by New England Peptide as a custom order with the following sequence: [Cyc(23,39)]H2NPGQEHPNARKYKGANKKGLSKGCFGLKLDRIGSMSGLGC—OH, as described by Lorget et al., 2012. The purity was >95%.

Measurements of cGMP Production in Tibia Growth Plates Using cGi500

cGMP production in chondrocytes within intact growth plates was measured using tibias dissected from newborn mice (0-1 day old) that globally expressed one or two copies of the the cGi500 FRET sensor, as previously described (Shuhaibar et al., 2017).Tibias were dissected and cultured overnight on Millicell membranes in BGJb medium with 0.1% bovine serum albumin, 100 units/ml of penicillin, and 100 μg/ml of streptomycin. In preparation for imaging, each tibia was slit to remove the tissue overlying the growth plate. Where indicated, the tibia was incubated in LB-100, cantharidin, or control medium, followed by addition of FGF18 (0.5 μg/ml+1 μg/ml heparin) or control medium containing heparin only. The tibia was then placed in a perfusion slide and the growth plate was imaged on the stage of a confocal microscope, as previously described (Shuhaibar et al., 2017).

### Rib Chondrocyte Cultures

Rib cages were dissected from newborn (0-2 day old) mice, and trimmed to remove the skin, spinal cord, and soft tissue around the sternum and ribs. Non-chondrocyte tissue was digested away by incubating the rib cages in 2 mg/ml pronase in PBS for one hour in a shaking water bath at 37° C., and then incubated in 3 mg/ml collagenase D in medium for one hour. After washing, the rib cages were transferred to a dish with fresh collagenase D and incubated for 5-6 hours, with trituration at 2 hours, to release the chondrocytes. The isolated cells were passed through a 40 um nylon cell strainer, resuspended in DMEM/F12 medium with 10% fetal bovine serum, 100 units/ml of penicillin, and 100 μg/m1 of streptomycin, and plated in 35 mm tissue culture dishes, at a cell density corresponding to one newborn mouse per plate. The cells were cultured for 3 days, at which point they were ˜75-90% confluent, then washed with PBS and incubated in serum-free medium for 18 hours. The cells were then incubated in LB-100 (10 μM), or control medium, followed by addition of FGF18 (0.5 ug/ml+1 μg/ml heparin) or control medium containing heparin only.

At end of the incubation period, dishes were washed in PBS and cells lysed in 250 μl of 1% SDS containing 10 mM sodium fluoride, 1 μM microcystin, and Roche protease inhibitor cocktail. Protein content was determined by a BCA assay. The protein yield per newborn mouse was ˜200-300 μg.

### Phostag Gel Electrophoresis, and Western Blotting

Proteins were separated in a Phos-tag-containing gel, as previously described (Egbert et al., 2014), except that chondrocyte lysates (50 μg protein) were used without immunoprecipitation. Blots were probed with an antibody that was made in guinea pig against the extracellular domain of mouse NPR2 (Ter-Avetisyan et al., 2014). This antibody was a gift from Hannes Schmidt (University of Tubingen), and has been previously validated for western blots (Robinson et al., 2017).

Ex vivo culture of fetal femurs

Femurs were cultured ex vivo, as described previously (Jonquoy et al., 2012; Lorget et al., 2012). The left femur was cultured in the presence of LB-100 (10 μM), BMN-111 (0.1 μM), or LB-100 (10 μM)+BMN-111 (0.1 μM), and compared with the non-treated right femur. The bone's length was measured on day 0 (D0) and day 6 (D6). Images were captured with an Olympus SZX12 stereo microscope and quantified using cellSens software (Olympus). The results were expressed as the increase in femur length or area (D6-D0) in the presence or absence of LB-100, BMN-111, or LB-100+BMN-111.

### Histology

Fetal femur (E16.5) explants were fixed in 4% paraformaldehyde, decalcified with EDTA (0.4 M), and embedded in paraffin. Serial 5 μm sections were stained with hematoxylin-eosin-safran (HES) reagent, using standard protocols. For immunohistochemical assessment, sections were labeled with the following antibodies and a Dako Envision Kit: anti-COL X (BIOCYC, N.2031501005; 1:50 dilution), anti-phosphorylated ERK1-2 (Thr180/Tyr182) (Cell Signaling Technology, #4370; 1:100 dilution), and anti-S0X9 (polyclonal antibody, Santa Cruz Biotechnology Inc., catalog D0609; dilution 1:75). Images were captured with an Olympus PD70-IX2-UCB microscope and quantified using cellSens software.

For analysis of the effect of the drug treatments on the area occupied by proliferative chondrocytes, these cells were identified by their round or columnar shape as seen with HES staining, and by the absence of collagen X labeling. We measured the total area occupied by chondrocytes within the whole growth plate and the area occupied by COLX-positive chondrocytes. The area for proliferating chondrocytes was calculated by subtracting the COLX-positive area from the whole growth plate area.

### Statistics

Data were analyzed using Prism 6 or higher (GraphPad Software). To compare more than two groups, we used one-way ANOVA followed by two-tailed t-tests with the Holm-Sidak correction for multiple comparisons, or two-way ANOVA followed by Sidak's multiple comparisons tests. Two groups were compared using either paired or unpaired two-tailed t-tests, as indicated in the figure legends.

### Results

### LB-100 Counteracts the Inactivation of NPR2 by FGF in Growth Plate Chondrocytes.

NPR2 activity in chondrocytes of intact growth plates was measured as previously described, using mice expressing a FRET sensor for cGMP, cGi500 (Shuhaibar et al., 2017). Tibias were isolated from newborn mice, and the overlying tissue was excised to expose the growth plate for confocal imaging (Data not shown). When the NPR2 agonist C-type natriuretic peptide (CNP) was perfused across the growth plate, the CFP/YFP emission ratio from cGi500 increased, indicating an increase in cGMP, due to stimulation of the guanylyl cyclase activity of NPR2 (Data not shown). Perfusion of A-type natriuretic peptide (ANP), which activates the NPR1 guanylyl cyclase, or perfusion of a nitric oxide donor (DEA/NO), which activates soluble guanylyl cyclases, did not increase cGMP (Data not shown), showing that among the several mammalian guanylyl cyclases, only NPR2 is active in the chondrocytes of the mouse growth plate. As previously shown, exposure of the growth plate to FGF18 suppressed the cGMP increase in response to CNP perfusion (Data not shown), indicating that FGF receptor activation decreases NPR2 activity.

Based on previous evidence that a PPP family phosphatase inhibitor, cantharidin (100 μM), inhibits the inactivation of NPR2 in growth plate chondrocytes by FGF (Shuhaibar et al., 2017), we tested a cantharidin derivative, LB-100, for which a phase I clinical trial indicated little toxicity (Chung et al., 2017). LB-100 is a catalytic inhibitor of PPP2 and PPPS, with IC50 values of 0.39±0.013 μM and 1.82±0.093 μM respectively, when tested in vitro with DiFMUP as the substrate (D'Arcy et al., 2019). LB-100 also inhibits PPP1, with an IC50 of 1.80±0.022 μM when tested under these same conditions (Richard Honkanen, personal communication). Based on its similarity to cantharidin, 10 μM LB-100 is also likely to inhibit PPP6, but not PPP4 or PPP7 (Swingle and Honkanen, 2019).

To investigate if LB-100 counteracts the inactivation of NPR2 by FGF, we used the following protocol:Tibias expressing cGi500 were preincubated with solutions of 1, 5 or 10 μM LB-100 for 60 minutes. FGF was then added and the incubation was continued for an additional 80 minutes. Following these incubations, the tibia was placed in a perfusion slide for confocal imaging, and cGMP production by NPR2 was monitored by measuring the increase in CFP/YFP emission ratio in response to CNP. Incubation with 10 μM LB-100 caused no visible change in chondrocyte morphology as imaged in the live growth plate by confocal microscopy, indicating no obvious toxicity (Data not shown).

After FGF treatment, the cGMP increase in response to CNP was small (Data not shown). However, when the tibia was preincubated with 5 or 10 μM LB-100 before applying FGF, the CNP-induced cGMP increase was enhanced (Data not shown). 1μM LB-100 had no effect. The CFP/YFP emission ratio attained after CNP perfusion in tibias that had been incubated in 5 or 10 μM LB-100 before the FGF treatment was similar to or greater than that in control tibias without FGF (Data not shown). The CNP-stimulated increases in the CFP/YFP emission ratio from cGi500 under these various conditions, and demonstrates that LB-100 counteracts the inactivation of NPR2 by FGF. LB-100 was more effective than cantharidin, with 5 μM LB-100 resulting in a stimulation equivalent to that seen with 10 μM cantharidin (Data not shown).

### LB-100 Counteracts the FGF-induced Dephosphorylation of NPR2 by FGF in Primary Chondrocyte Cultures

To investigate if LB-100 Counteracts the FGF-induced Dephosphorylation of NPR2, we used Phostag gel electrophoresis (Kinoshita et al., 2009) to analyze the phosphorylation state of NPR2 in isolated chondrocytes from the ribs of newborn mice. After 4 days in culture, the chondrocytes had formed a monolayer with a cobblestone appearance (Data not shown). A 1 hour incubation with 10 μM LB-100 did not cause any visible change in cell morphology (Data not shown). We then compared the phosphorylation state of NPR2 in chondrocytes with and without LB-100 preincubation, and with and without subsequent exposure to FGF. Chondrocyte proteins were separated by Phos-tag gel electrophoresis, which retards migration of phosphorylated proteins, and western blots were probed for NPR2 (Data not shown). Without

FGF treatment, NPR2 protein from the rib chondrocytes was present in a broad region of the gel. With FGF treatment, the ratio of the signal in the upper vs lower regions decreased (Data not shown), indicating NPR2 dephosphorylation in response to FGF and confirming, with primary chondrocytes, a previous study using a rat chondrosarcoma (RCS) cell line (Robinson et al., 2017). However, if the chondrocytes were preincubated with 10 μM LB-100, the dephosphorylation in response to FGF was only partial, indicating that LB-100 counteracts the FGF-induced dephosphorylation of NPR2 (Data not shown).

### In Fgfr3^{Y367C/+} Femurs, LB-100 Enhances the Stimulation of Bone Growth by the Hydrolysis Resistant NPR2 Agonist BMN-111.

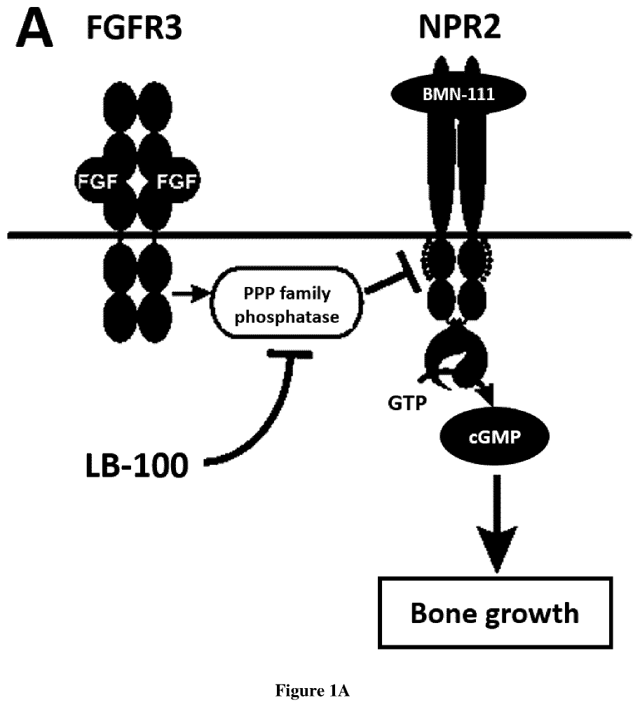

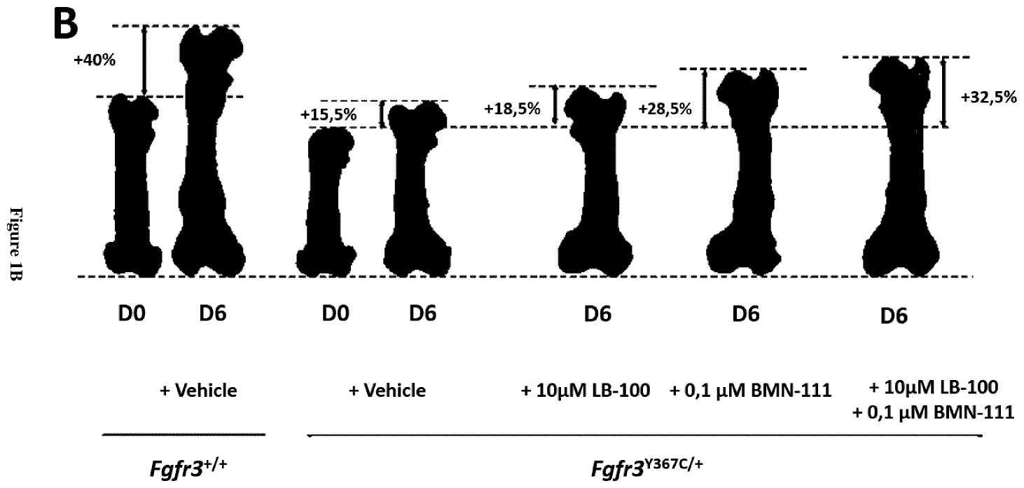

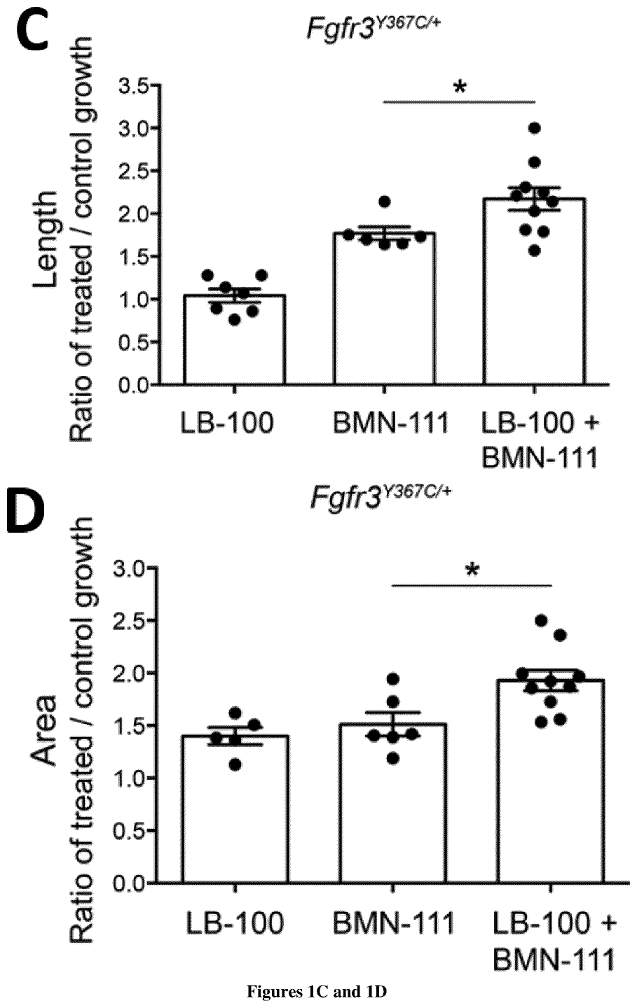

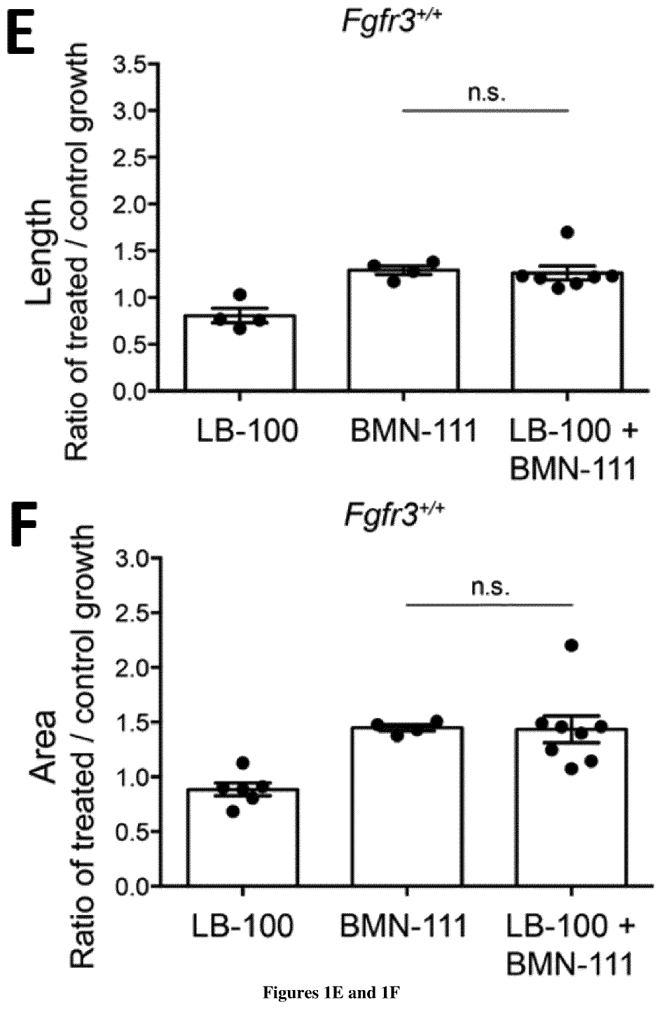

Previously we showed that the hydrolysis-resistant CNP analog BMN-111 increases bone growth in a mouse model of achondroplasia in which tyrosine 367 is changed to a cysteine (Fgfr3^{Y367C/+}), resulting in constitutive activation of FGFR3 (Pannier et al., 2009; Lorget et aL, 2012). However, BMN-111 only partially rescued the effect of the FGFR3-activating mutation. Our finding that LB-100 opposes the FGF inhibition of NPR2 activity in chondrocytes suggested that applying LB-100 together with BMN-111 might enhance the stimulation of growth (FIG. 1A). Like CNP, BMN-111 (0.1 μM) stimulated NPR2 activity in growth plate chondrocytes (Data not shown).

As previously reported (Lorget et al., 2012), 0.1 μM BMN-111 increased the rate of elongation of cultured femurs from embryonic day 16.5 Fgfr3^{Y367C/+} mice (FIGS. 1B, 1C, and Data not shown). The mean rate of increase in bone length in the BMN-111-stimulated Fgfr3^{Y367C/+} femurs was 1.77 times that in vehicle-treated bones (FIG. 1C). LB-100 alone did not significantly increase the rate of bone elongation, showing a growth rate ratio of 1.04 for LB-100/control (FIGS. 1C). However, when Fgfr3^{Y367C/+} femurs were cultured with BMN-111 together with 10 μM LB-100, the mean rate of bone elongation increased to 2.17 times that in untreated bones (FIGS. 1C). Thus, the combination of BMN-111 and LB-100 significantly increased the bone elongation rate to a level —23% higher than that with BMN-111 alone.

In addition to these measurements of bone length, we also measured the effect of LB-100 and BMN-111 on the rate of increase of the total bone and cartilage area (Data not shown). Based on these area measurements, LB-100 by itself stimulated growth of femurs from Fgfr3^{Y367C/+} mice, as did BMN-111 by itself, with a growth rate ratio of 1.40 for LB-100/control, and a growth rate ratio of 1.51 for BMN-111/control (FIGS. 1D). The combination of LB-100 and BMN-111 was even more effective, with a growth rate ratio of 1.93. Thus, the combination of BMN-111 and LB-100 enhanced the rate of increase in area by 27% compared with BMN-111 alone (FIG. 1D).

With cultured femurs from Fgfr3^{+/+} mice, BMN-111 increased the rate of bone growth, but combining BMN-111 with LB-100 did not enhance the growth rate beyond that seen with BMN-111 alone (FIGS. 1E and 1F). This result contrasts with the ability of LB-100 to enhance BMN-111-stimulated bone growth in Fgfr3^{Y367C/+} mice. Because Fgfr3^{Y367C/+} mice have elevated FGFR3 tyrosine kinase activity due to an activating Fgfr3 mutation (Gibbs and Legeai-Mallet, 2007), their NPR2 would be expected to be less phosphorylated and less active, and LB-100 would be expected to restore their NPR2 phosphorylation and activity towards Fgfr3^{+/+} levels, thus increasing bone growth. In contrast, if baseline NPR2 phosphorylation is higher in Fgfr3^{+/+} vs Fgfr3^{Y367C/+} mice, the growth stimulating effect of LB-100 might be less.

Combined Treatment with LB100 and BMN111 Improves Growth Plate Cartilage Homeostasis.

Histological analyses of the epiphyseal growth plates of femurs showed that combining BMN-111 and LB-100 treatment modified the cartilage growth homeostasis in both proximal and distal growth plates (Data not shown). Prehypertrophic and hypertrophic chondrocytes produce an extracellular matrix rich in Collagen type X (COLX), allowing us to use COLX immunostaining to label the hypertrophic region, and to visualize and measure individual cells. This labeling revealed a highly beneficial effect of the combined treatment on the size of the cells in the hypertrophic area of Fgfr3^{Y367C/+} femurs (FIG. 2). Compared to the Fgfr3+/+ growth plates, the mean cross-sectional areas of the hypertrophic chondrocytes in Fgfr3^{Y367C/+} distal and proximal growth plates were reduced by about half (FIG. 2). As previously reported (Lorget et al., 2012), BMN-111 increased the size of the Fgfr3^{Y367C/+} hypertrophic chondrocytes, but the cells were smaller than for the wildtype (FIG. 2). However, with the combined treatment of BMN-111 and LB-100, the mean area of the Fgfr3^{Y367C/+} hypertrophic cells in the proximal growth plate was 32% greater than with BMN-111 alone, and similar to that of Fgfr3+/+ hypertrophic cells, indicating that the final differentiation of the chondrocytes was restored by the treatment (FIG. 2). Under the conditions used for this analysis, the improvement was only significant in the proximal growth plate (FIG. 2), in which chondrocyte development precedes that in the distal growth plate during the endochondral growth process (Data not shown).We also observed a beneficial effect of the combined treatment on the proliferative region of the growth plate. We measured the area of the proliferative region by subtracting the hypertrophic area, identified by COLX labeling, from the total growth plate area. Based on these measurements, the combined treatment increased the total proliferative growth plate area of the femur by an average of 33% over vehicle, compared to 20% for BMN-111 alone (Data not shown). Thus, the combined treatment increased the proliferative area by 13% compared to BMN-111 alone (Data not shown). In summary, the combined treatment both increased the proliferative growth plate area of the femur and restored chondrocyte terminal differentiation. CNP signaling through NPR2 in the growth plate inhibits the MAP kinase pathway and its extracellular signal-regulated kinase 1 and 2 (ERK1/2) (Ozasa et al., 2005; Krejci et al., 2005). Therefore, we investigated the impact of treatment with LB-100+BMN-111 on the phosphorylation of ERK1/2. In agreement with the constitutive activity of the Y367C Fgfr3 gain-of-function mutation acting to decrease NPR2 activity (Data not shown), we observed by immunostaining a high level of phosphorylated ERK1/2 in the proximal and distal parts of the cartilage compared to controls (Data not shown). The combined LB-100 and BMN-111 treatment decreased the activity of the MAP kinase pathway as demonstrated by the decreased phosphorylation of ERK1/2 in the proximal and distal growth plates of the femurs (Data not shown). These data are in agreement with 1) the role of the MAP kinase pathway as a regulator of chondrocyte differentiation and with 2) our hypothesis that the elevation of cGMP inhibits the MAP kinase pathway thus promoting bone growth. Lastly, we examined the expression of the SOX9, a master transcription factor that is upregulated in Fgfr3 gain-of-function mouse models (Ornitz and Legeai-Mallet, 2017). The activation of ERK1/2 increases SOX9 expression, which functions to suppress chondrocyte terminal differentiation (Lefebvre and Dvir-Ginzberg, 2017; Murakami et al., 2000). Consistent with these findings, we observed that the activation of FGFR3 signaling in the Fgfr3^{Y367C/+} femurs increased the expression of SOX9, relative to the Fgfr3+/+ control. SOX9 protein was seen to accumulate visibly in the proliferative and hypertrophic zones of the growth plate cartilage (Data not shown). Treatment of Fgfr3^{Y367C/+} femurs with BMN-111+LB-100 reduced SOX9 expression in proximal and distal growth plates, and its expression returned to a more normal level, closer to that of the Fgfr3^{+/+} femurs. The fact that SOX9 growth plate expression was decreased confirms the beneficial action of BMN111+LB100 treatment on growth plate cartilage homeostasis.

### EXAMPLE 2: IN VIVO

### Material & Methods

### The Mouse Model

The Fgfr3^{Y367C/+} mouse model has been described previously (Pannier et al 2009). All the mice had a C57BL/6 background. Cartilage and bone analyses were performed on 16-day-old mice. The Fgfr3^{Y367C/+} mice were 1-day old upon treatment initiation, and received daily subcutaneous administrations of LB100 (1 mg.kg−1 body weight)+BMN111 (800 ug.kg−1 body weight) or vehicle (3.5 mM HCl, 0.1% DMSO) for 2-weeks. Long bones were measured using a caliper (VWRi819-0013, VWR International).

### Combined Drug Treatment

BMN-111 was synthesized by New England Peptide as a custom order with the following sequence: [Cyc(23,39)]H2NPGQEHPNARKYKGANKKGLSKGCFGLKLDRIGSMSGLGC—OH, as described by Lorget et al., 2012. The purity was >95%.

LB100 was synthesized by Selleckchem with the following sequence (3-(4-methylpiperazine-1-carbonyl)-7-oxabicyclo[2.2.1]heptane-2-carboxylic acid) as described by Shuhaibar L C et al 2021.

### Results

### Combined Treatment (LB100+BMN11) Modulates Bone Growth in the Fgfr3 Mouse Model of ACH

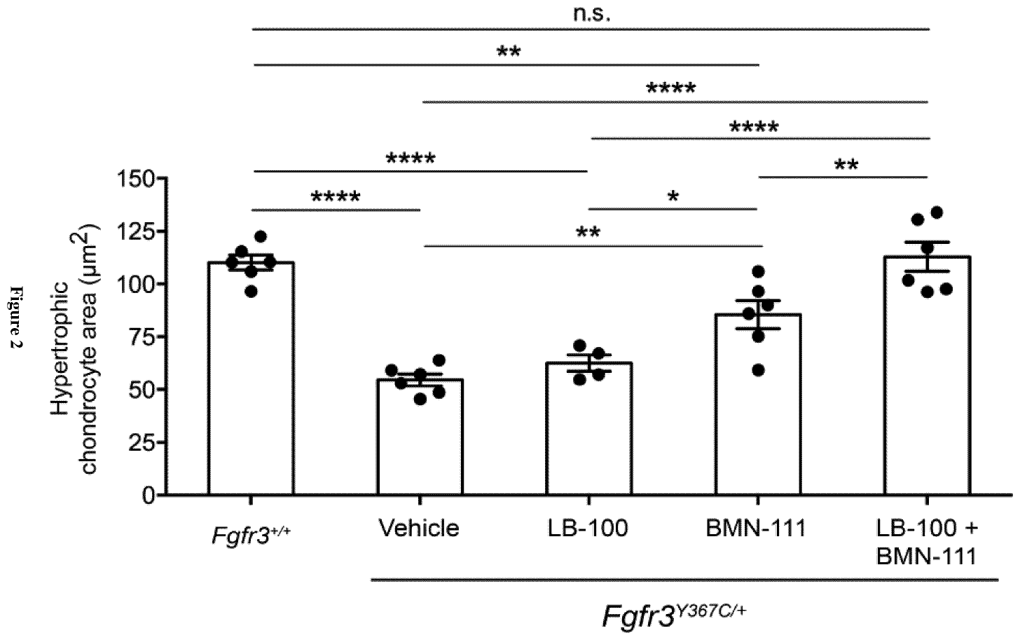

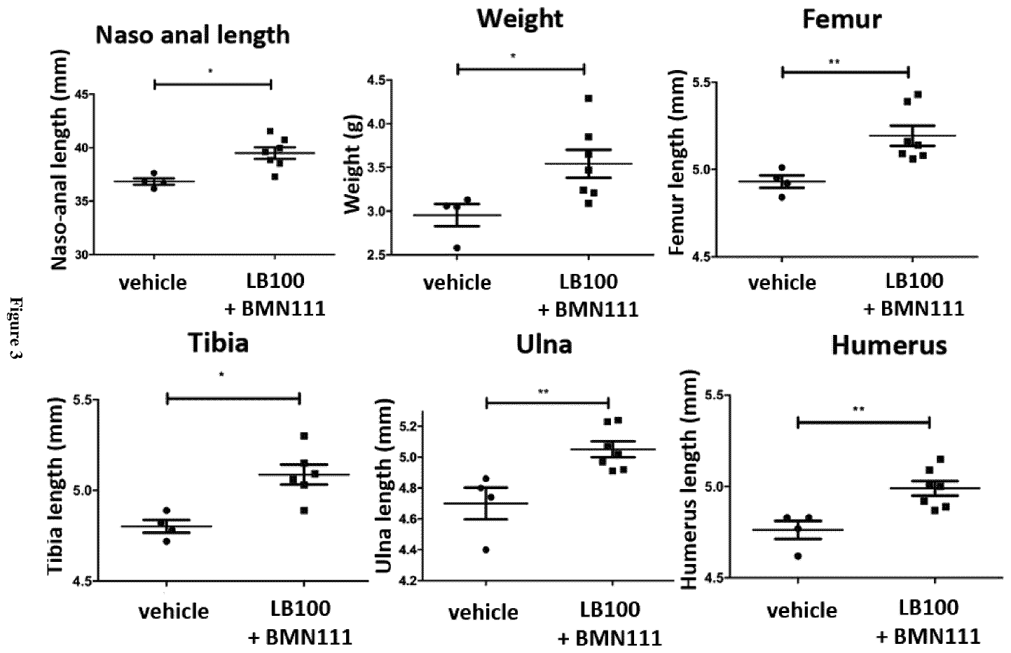

To confirm LB100+BMN-111 in vivo effect on bone growth, we next sought to determine whether or not a combination of BMN-111 and LB-100 could regulate bone growth in the Fgfr3^{Y367C/+} murine model of ACH. The Fgfr3^{Y367C/+} mice were 1 day old when treatment began and received once-daily subcutaneous administrations of LB-100 (1 mg.kg−1 body weight)+BMN-111 (800 ug.kg−1 body weight) or vehicle for 2-weeks. Significant improvement in dwarfism was noted after only 2 weeks of treatment. The mean naso-anal length was 7.24% (p<0.05)) greater in treated animals than in controls. Likewise, the mean weight was 19.89% (p<0.05) greater in treated animals (FIG. 3). We did not observe any adverse events (e.g. weight loss or death). With regard to long bone growth, the femur and tibia were respectively 5.38% (p<0.01) and 4.82% (p<0.05) longer in treated mice than in controls. Similarly, the humerus and ulna in treated mice were respectively 4.76% (p<0.01) and 7.5% (p<0.01) longer than in non-treated controls (FIG. 3).

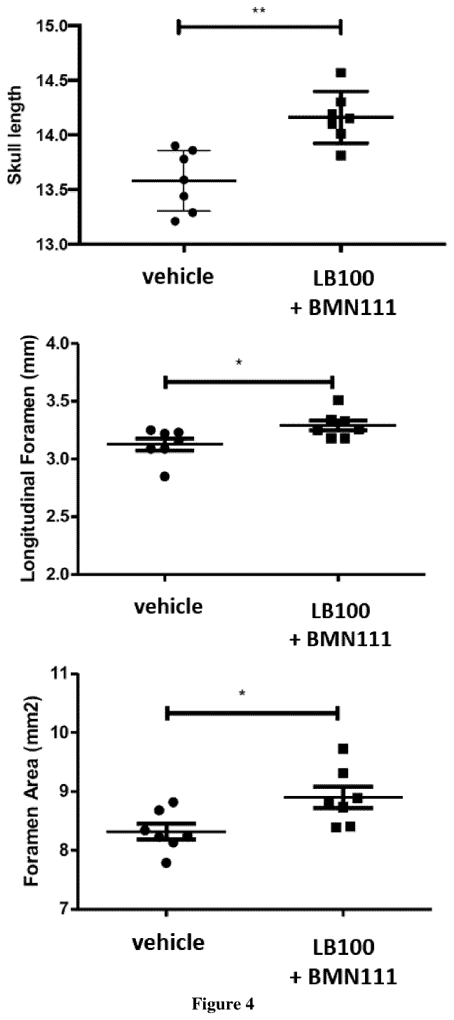

To visualize combined treatment's in vivo on skull and foramen magnum at the skull base, we analyzed KTScanner. We observed that the mean transversal length and longitudinal length of the skull were respectively 3.89% (p<0.05) and 5.30% (p<0.05) greater in treated animals and the foramen magnum area were higher 5.6% (p<0.05) in treated animals (FIG. 4).

To characterize combined treatment's in vivo mode of action, we analyzed the growth plate. The treatment strongly modified the structural organization of the Fgfr3^{Y367C/+} growth plate cartilage and restored the delay of secondary ossification center formation in treated mice (Data not shown). We observed an enlargement of the epiphysis of the proximal part of the femur in treated animals versus untreated, the collagen type X labelling pointed out this change of the epiphysis (Data not shown). A large area of collagen type Xis visible in all animals treated by LB100+BMN-111. As with the ex vivo experiments, and in order to assess combined treatment's in vivo effect on the regulators of chondrocyte differentiation that are perturbed in ACH, we evaluated the expression levels of phosphorylation levels of Erk1-2 (Data not shown). As observed in ex vivo femur cultures, the abnormal in vivo overexpression of phosphorylated Erk1/2 had normalized after combined treatment (Data not shown).

These in vivo results demonstrate that the combination LB100+BMN-111 can be used to control long bone elongation in FGFR3-related disease.

### Conclusion

Understanding of the mechanisms used by FGFR3 and CNP as important regulators of longitudinal bone growth has allowed the development of an effective therapeutic strategy using a CNP analog (vosoritide, also known as BMN-111) to treat achondroplasia (Savarirayan et al., 2019). The findings described here identify the PPP family phosphatase inhibitor LB-100 as a stimulator of bone growth when used in combination with this CNP analog to stimulate production of cGMP by NPR2. Firstly, using isolated bones incubated with FGF to mimic an achondroplasia-like condition, we show that pretreatment with LB-100 counteracts the decrease in NPR2 guanylyl cyclase activity by FGFR3. Secondly, our results support the hypothesis that FGFR3 acts by dephosphorylating NPR2 in chondrocytes and that LB-100 suppresses the dephosphorylation. Moreover, application of a combination of BMN-111 and LB-100 to bones from the achondroplasia mouse model Fgfr3^{Y367C/+} results in growth that exceeds that stimulated by BMN-111 alone, demonstrating the therapeutic potential of this combination treatment for skeletal dysplasias such as achondroplasia.

Our data also show the benefit of this treatment for growth plate cartilage during bone development in Fgfr3^{Y367C/+} mice. During the process of endochondral ossification, chondrocytes actively proliferate in the resting and proliferating chondrocyte zone, and then differentiate to hypertrophic chondrocytes, which lose the capacity to proliferate. The terminally differentiated hypertrophic cells are removed by cell death or transdifferentiate into osteoblasts. It is well known that FGFR3 signaling decreases bone growth by inhibiting both chondrocyte proliferation and differentiation and bone formation. SOX9 is known to be required to permit proliferation and differentiation, which are the regulators (drivers) of bone elongation (Lefebvre and Dvir-Ginzberg, 2017), and it has been proposed that FGFR3 uses ERK1/2 to restrict hypertrophic differentiation (Murakami et al., 2004). Here, we showed that the treatment with BMN-111 and LB-100 reduced the levels of phosphorylated ERK1/2 and SOX9, thus modifying chondrocyte differentiation and allowing bone growth. In addition, we noted an impressive increase in the size of the hypertrophic cells. We concluded that the treatment perfectly restored cartilage homeostasis, and we hypothesize that the elevated cGMP resulting from this treatment could be a key regulator of transdifferentiation of hypertrophic cells into osteoblasts and could control the chondrogenic or osteogenic fate decision.

The increase in NPR2 phosphorylation by LB-100 is correlated with improved chondrocyte proliferation in Fgfr3^{Y367C/+} femurs, consistent with previous results with a mouse model (Npr2-7E) mimicking constitutive phosphorylation of NPR2 (Shuhaibar et al., 2017). LB-100 inhibits multiple PPP family phosphatases (D'Arcy et al., 2019), and thus although our results provide a proof of principle for a possible combination treatment, a more specific phosphatase inhibitor would be more optimal. Determination of the particular phosphatase(s) that dephosphorylate NPR2 in chondrocytes, and development of more specific inhibitors of these phosphatases, could lead to future therapies.

Recent mouse studies indicate that in addition to increasing prepubertal bone elongation, phosphorylation of NPR2 increases bone density, due to an increase in the number of active osteoblasts at the bone surface (Robinson et al., 2020). Because low bone density is one of the key clinical features of achondroplasia (Matsushita et al., 2016), the combination a CNP analog and a phosphatase inhibitor could also have a beneficial impact on bone density for patients with achondroplasia and related conditions. In addition, this treatment could have potential for treatment of osteoporosis. Lastly, because CNP/NPR2 also plays a key role in regulation of joint homeostasis, inactivation of ERK1/2 due to elevated cGMP could also be beneficial for preventing or minimizing cartilage loss and promoting repair of the damaged articular cartilage in skeletal disorders and osteoarthritis, an extremely common disease of adulthood (Peake et al., 2014). More generally, the combination of natriuretic peptides and phosphatase inhibitors could have therapeutic potential for multiple disorders involving NPR2 and the related guanylyl cyclase NPR1 that also requires phosphorylation for activity (Kuhn, 2016).

In summary, the combined (LB-100+BMN-111) treatment acts on both chondrocyte proliferation and differentiation, thus promoting better bone growth. In achondroplasia, the homeostasis of the growth plate is disturbed, and proliferation and differentiation are affected by the overactivation of FGFR3. Currently, BMN111 (vosoritide) is being studied in children with ACH, and as demonstrated in preclinical studies, BMN-111 mostly restores the defective differentiation in the growth plate (Lorget et al., 2012). Recently reported phase 2 data demonstrated that vosoritide resulted in a sustained increase in annualized growth velocity for up to 42 months in children 5 to 14 years of age with achondroplasia (Savarirayan et al., 2019). In this study, we described a combination treatment that could increase bone growth rate to a higher level than vosoritide alone, could shorten the required time of treatment, and could be considered as intermittent treatment for patients with achondroplasia.

### REFERENCES

Throughout this application, various references describe the state of the art to which this invention pertains. The disclosures of these references are hereby incorporated by reference into the present disclosure.

Baena V, et al. Cellular heterogeneity of the LH receptor and its significance for cyclic GMP signaling in mouse preovulatory follicles [published online May 8, 2020]. Endocrinology. https://doi: 10.1210/endocr/bqaa074.

Bartels C F, et al. Mutations in the trans-membrane natriuretic peptide receptor NPR-B impair skeletal growth and cause acromesomelic dysplasia, type Maroteaux. Am J Hum Genet. 2004; 75(1):27-34.

Bocciardi R, et al. Overexpression of the C-type natriuretic peptide (CNP) is associated with overgrowth and bone anomalies in an individual with balanced t(2; 7) translocation. Hum Mutat. 2007; 28(7):724-731.

Chen L, et al. Gly369Cys mutation in mouse FGFR3 causes achondroplasia by affecting both chondrogenesis and osteogenesis. J Clin Invest. 1999; 104(11):1517-1525.

Chung V, et al. Safety, tolerability, and preliminary activity of LB-100, an inhibitor of protein phosphatase 2A, in patients with relapsed solid tumors: an open-label, dose escalation, first-in-human, phase I trial. Clin Cancer Res. 2017; 23(13):3277-3284.

Chusho C, et al. Dwarfism and early death in mice lacking C-type natriuretic peptide. Proc Natl Acad Sci USA. 2001; 98(7):4016-4021.

Colvin J S, Bohne B A, Harding G W, McEwen D G, Ornitz D M. Skeletal overgrowth and deafness in mice lacking fibroblast growth factor receptor 3. Nat Genet. 1996; 12(4):390-397.

Cui J, et al. Inhibition of PP2A with LB-100 enhances efficacy of CAR-T cell therapy against glioblastoma. Cancers (Basel). 2020; 12(1):139.

D'Arcy B M, et al. The antitumor drug LB-100 is a catalytic inhibitor of protein phosphatase 2A (PPP2CA) and 5 (PPPSC) coordinating with the active-site catalytic metals in PPPSC. Mol Cancer Ther. 2019; 18(3):556-566.

Deng C, Wynshaw-Boris A, Zhou F, Kuo A, Leder P. Fibroblast growth factor receptor 3 Is a negative regulator of bone growth. Cell. 1996; 84(6):911-921.

Egbert J R, et al. Dephosphorylation and inactivation of the NPR2 guanylyl cyclase in the granulosa cells contributes to the LH-induced cGMP decrease that causes resumption of meiosis in rat oocytes. Development. 2014; 141(18):3594-3604.

Geister K A, et al. A novel loss-of-function mutation in Npr2 clarifies primary role in female reproduction and reveals a potential therapy for acromesomelic dysplasia, Maroteaux type. Hum Mol Genet. 2013; 22(2):345-357.

Gibbs L, Legeai-Mallet L. FGFR3 intracellular mutations induce tyrosine phosphorylation in the Golgi and defective glycosylation. Biochim Biophys Acta. 2007; 1773(4):502-512.

Ho W S, et al. Pharmacologic inhibition of protein phosphatase-2A achieves durable immune-mediated antitumor activity when combined with PD-1 blockade. Nat Commun.

Jonquoy A, et al. A novel tyrosine kinase inhibitor restores chondrocyte differentiation and promotes bone growth in a gain-of-function Fgfr3 mouse model. Hum Mol Genet. 2012; 21(4): 841-851.

Khan S, Ali R H, Abbasi S, Nawaz M, Muhammad N, Ahmad W. Novel mutations in natriuretic peptide receptor-2 gene underlie acromesomelic dysplasia, type Maroteaux. BMC Med Genet. 2012; 13:44.

Kinoshita E, Kinoshita-Kikuta E, Koike T. Separation and detection of large phosphoproteins using Phos-tag SDS-PAGE. Nat Protoc 2009; 4(10):1513-1521.

Krejci P, et al. Interaction of fibroblast growth factor and C-natriuretic peptide signaling in regulation of chondrocyte proliferation and extracellular matrix homeostasis. J Cell Sci. 2005; 118(21):5089-5100.

Kuhn M. Molecular physiology of membrane guanylyl cyclase receptors. Physiol Rev. 2016; 96(2):751-804.

Lai D, et al. PP2A inhibition sensitizes cancer stem cells to ABL tyrosine kinase inhibitors in BCR-ABL+ human leukemia. Sci Transl Med. 2018; 10(427):8735.

Lefebvre V, Dvir-Ginzberg M. SOX9 and the many facets of its regulation in the chondrocyte lineage. Connect Tissue Res. 2017; 58(1):2-14.

Legeai-Mallet L, Benoist-Lasselin C, Munnich A, Bonaventure J. Overexpression of FGFR3, Stat1, StatS and p21Cip1 correlates with phenotypic severity and defective chondrocyte differentiation in FGFR3-related chondrodysplasias. Bone. 2004; 34(1):26-36.

Li C, Chen L, Iwata T, Kitagawa M, Fu X Y, Deng C X. A Lys644Glu substitution in fibroblast growth factor receptor 3 (FGFR3) causes dwarfism in mice by activation of STATs and ink4 cell cycle inhibitors. Hum Mol Genet. 1999; 8(1):35-44.

Long F, Ornitz D M. Development of the endochondral skeleton. Cold Spring Harb Perspect Biol. 2013; 5(1):a008334.

Lorget F, et al. Evaluation of the therapeutic potential of a CNP analog in a Fgfr3 mouse model recapitulating achondroplasia. Am J Hum Genet. 2012; 91(6):1108-1114.

Maroteaux P, Martinelli B, Campailla E. Le nanisme acromésomélique. Presse Med. 1971; 79(42): 1839-1842.

Matsushita M, et al. Low bone mineral density in achondroplasia and hypochondroplasia. Pediatr Int. 2016; 58(8):705-708.

Miura K, et al. Overgrowth syndrome associated with a gain-of-function mutation of the natriuretic peptide receptor 2 (NPR2) gene. Am J Med Genet A. 2014; 164A(1):156-163.

Moncla A, et al. A cluster of translocation breakpoints in 2q37 is associated with overexpression of NPPC in patients with a similar overgrowth phenotype. Hum Mutat. 2007; 28(12):1183-1188.

Murakami S, Kan M, McKeehan W L, de Crombrugghe B. Up-regulation of the chondrogenic Sox9 gene by fibroblast growth factors is mediated by the mitogen-activated protein kinase pathway. Proc Natl Acad Sci USA. 2000; 97(3)1113-1118.

Murakami S, Balmes G, McKinney S, Zhang Z, Givol D, de Crombrugghe B. Constitutive activation of MEK1 in chondrocytes causes Statl-independent achondroplasia-like dwarfism and rescues the Fgfr3-deficient mouse phenotype. Genes Dev. 2004; 18(3):290-305.

Nakao K, et al. The local CNP/GC-B system in growth plate is responsible for physiological endochondral bone growth. Sci Rep. 2015; 5:10554.

Naski M C, Colvin J S, Coffin J D, Ornitz D M. Repression of hedgehog signaling and BMP4 expression in growth plate cartilage by fibroblast growth factor receptor 3. Development. 1998; 125(24):4977-4988.

Ornitz D M, Itoh N. The fibroblast growth factor signaling pathway. Wiley Interdiscip Rev Dev Biol. 2015; 4(3):215-266.

Ornitz D M, Legeai-Mallet L. Achondroplasia: development, pathogenesis, and therapy. Dev Dyn. 2017; 246(4):291-309.

Ozasa, A, et al. Complementary antagonistic actions between C-type natriuretic peptide and the MAPK pathway through FGFR-3 in ATDCS cells. Bone. 2005; 36(6):1056-1064.

Pannier S, et al. Activating Fgfr3 Y367C mutation causes hearing loss and inner ear defect in a mouse model of chondrodysplasia. Biochim Biophys Acta. 2009; 1792(2):140-147.

Pannier S, et al. Delayed bone age due to a dual effect of FGFR3 mutation in achondroplasia. Bone. 2010; 47(5):905-915.

Peake N J, et al. Role of C-type natriuretic pept ide signalling in maintaining cartilage and bone function. Osteoarthritis Cartilage. 2014; 22(11):1800-1807.

Potter L R. Phosphorylation-dependent regulation of the guanylyl cyclase-linked natriuretic peptide receptor B: dephosphorylation is a mechanism of desensitization. Biochemistry 1998; 37(8):2422-2429.

Potter L R. Regulation and therapeutic targeting of peptide-activated receptor guanylyl cyclases. Pharmacol Ther. 2011; 130(1):71-82.

Robinson J W, Egbert J R, Davydova J, Schmidt H, Jaffe, L A, Potter L R. Dephosphorylation is the mechanism of fibroblast growth factor inhibition of guanylyl cyclase-B. Cell Signal. 2017; 40:222-229.

Robinson J W, et al. Male mice with elevated C-type natriuretic peptide-dependent guanylyl cyclase-B activity have increased osteoblasts, bone mass and bone strength. Bone 2020; 135:115320.

Rousseau F, et al. Mutations in the gene encoding fibroblast growth factor receptor-3 in achondroplasia. Nature 1994; 371(6494):252-254.

Savarirayan R, et al. C-type natriuretic peptide analogue therapy in children with achondroplasia. N Engl J Med. 2019; 381(1):25-35.

Shiang R, et al. Mutations in the transmembrane domain of FGFR3 cause the most common genetic form of dwarfism, achondroplasia. Cell 1994; 78(2):335-342.

Shuhaibar L C, et al. Dephosphorylation of juxtamembrane serines and threonines of the NPR2 guanylyl cyclase is required for rapid resumption of oocyte meiosis in response to luteinizing hormone. Dev Biol. 2016; 409(1):194-201.

Shuhaibar L C, et al. Dephosphorylation of the NPR2 guanylyl cyclase contributes to inhibition of bone growth by fibroblast growth factor. eLife. 2017; 4; 6:e31343.

Shuhaibar L C, Kaci N, Egbert J R, Horville T, Loisay L, Vigone G, Uliasz T F, Dambroise E, Swingle M R, Honkanen R E, Biosse Duplan M, Jaffe L A, Legeai-Mallet L. Phosphatase inhibition by LB-100 enhances BMN-111 stimulation of bone growth. JCI Insight. 2021 May 10; 6(9):141426.

Swingle M R, Honkanen R E. Inhibitors of serine/threonine protein phosphatases: biochemical and structural studies provide insight for further development. Curr Med Chem. 2019; 26(15):2634-2660.

Tamura N, Doolittle L K, Hammer R E, Shelton J M, Richardson J A, Garbers D L. Critical roles of the guanylyl cyclase B receptor in endochondral ossification and development of female reproductive organs. Proc Natl Acad Sci USA. 2004; 101(49):17300-17305.

Ter-Avetisyan G, Rathjen F G, Schmidt H. Bifurcation of axons from cranial sensory neurons is disabled in the absence of Npr2-induced cGMP signaling. J Neurosci. 2014; 34(3):737-747.

Thunemann M, et al. Transgenic mice for cGMP imaging. Circ Res. 2013; 113(4):365-371.

Tsuji T, Kunieda T. A loss-of-function mutation in natriuretic peptide receptor 2 (Npr2) gene is responsible for disproportionate dwarfism in Cn/Cn mouse. J Biol Chem. 2005; 280(14): 14288-14292.

Yasoda A, et al. Overexpression of CNP in chondrocytes rescues achondroplasia through a MAPK-dependent pathway. Nat Med. 2004; 10(1):80-86.

1. A method of treatment of FGFR3-related skeletal disease in a patient need thereof comprising administering to the patient a therapeutically effective amount of a combination of a phosphatase inhibitor and an NPR2 agonist.

2. A method of improving bone growth in a patient need thereof comprising in administering to the patient a therapeutically effective amount of a combination of a phosphatase inhibitor and a NPR2 agonist.

3. A method of increasing whole growth plate cartilage in a patient need thereof comprising administering to the patient a therapeutically effective amount of a combination of a phosphatase inhibitor and a NPR2 agonist.

4. The method according to claim 1, wherein the FGFR3-related skeletal disease is selected from the group consisting of thanatophoric dysplasia type I, thanatophoric dysplasia type II, severe achondroplasia with developmental delay and acanthosis nigricans, hypochondroplasia, achondroplasia and FGFR3-related craniosynostosis.

5. The method according to claim 1, wherein the FGFR3-related skeletal diseases is achondroplasia.

6. The method according to claim 1, wherein the phosphatase inhibitor is LB-100.

7. The method according to claim 1, wherein the NPR2 agonist is BMN-111.

8. The method of claim 1, wherein the phosphatase inhibitor and/or the NPR2 agonist are administered as active principles of a pharmaceutical composition comprising at least one pharmaceutically acceptable excipient.

9. The method of claim 4, wherein the FGFR3-related craniosynostosis is Muenke syndrome or Crouzon syndrome with acanthosis nigricans.
